(19) United States
(12) Patent Application Publication
(10) Pub. No.: US 2011/0245603 A1
(43) Pub. Date: Oct. 6, 2011

(54) LASER ENDOSCOPIC SURGICAL INSTRUMENT

(76) Inventor: JAMES K. BRANNON, LEAWOOD, KS (US)

(21) Appl. No.: 13/039,191

(22) Filed: Mar. 2, 2011

Related U.S. Application Data
(60) Provisional application No. 61/319,166, filed on Mar. 30, 2010.

(51) Int. Cl. A61B 1/00

(52) U.S. Cl. 600/108

(57) ABSTRACT

The present invention provides an apparatus and method for a laser sighting endoscopic instrument incorporating a laser unit for attachment to an endoscopic instrument in alignment with an axis of a lumen within a cannula portion of the instrument for visualizing a field of surgical access.

### CROSS-REFERENCE TO RELATED APPLICATION

This application claims the benefit of the prior filed U.S. non-provisional application No. 61/319,166 filed Mar. 2, 2010, the contents of which is incorporated herein by reference.

### FIELD OF THE INVENTION

The present invention is broadly directed to improvements in endoscopic surgery and, more particularly, to the use of a laser instrument to determine a field of surgical access such as within an endoscopic surgical site.

### BACKGROUND OF THE INVENTION

Modern surgery tends toward minimally invasive techniques whenever possible. Although often more complicated in some ways for the surgeon, minimally invasive techniques result in less trauma to the patient and less scarring because of much smaller incisions thereby promoting faster healing and reducing possibilities for infections. In general, minimally invasive surgeries involve making one or more small incisions at appropriate locations and inserting tubular devices through the incisions to the surgical site. The tubular devices may be referred to as endoscopes, arthroscopes, and the like and typically have optical fiber based optical viewing apparatus and light sources, surgical instruments, lumens for exchanging fluids with the surgical site, or combinations thereof extending therethrough. In some circumstances it is more appropriate to separate the light source and viewing scope from specifically surgical instruments, thus requiring two incisions and endoscopes. This technique is sometimes referred to as triangulation. In other instances, external types of imaging techniques are used for locating endoscopic instruments, such as fluoroscopes, computed tomography, magnetic resonance imaging, or the like.

Endoscopic instruments are configured in a number of different ways depending on their intended purpose. There are rigid endoscopes and flexible endoscopes. Rigid endoscopic instruments are preferred in situations when precise placement of an instrument is required, as for a surgical procedure. Some endoscopes are simply tubes or portal instruments which provide access to a surgical site for instruments which are passed through the scopes or for the exchange of fluids to and from the surgical site. Viewing scopes, including light sources, may be used for viewing a surgical site for diagnostic purposes or to view surgical operations occurring through the same scope or a different scope. Surgical operations may include cutting, shaving, debriding, cauterizing, or the like as well as grasping tissues or parts of organs, such as with forceps.

In the use of endoscopic instruments, it is often necessary for the surgeon to locate the surgical site indirectly, that is, without a direct view of the site initially. This is especially true with regard to arthroscopic surgery. It has often been necessary to employ radiopaque endoscopic instruments and a radiant imaging technique, such as fluoroscopy or computed tomography to extend an endoscopic instrument from an external incision to the surgical site. Direct viewing of an anatomical site is often necessary for more conclusive diagnosis of the condition of tissue at the site and for surgical intervention or treatment at the site. In the case of arthroscopic or endoscopic surgery, direct viewing is sometimes accomplished using a second endoscopic instrument which enters at a different angle from a scope through which a surgical instrument will be passed. This is referred to as triangulation.

A problem which sometimes occurs, especially in hip joint surgery, is that the field of view greatly exceeds the field of surgical access, that is, the range of motion available to the surgeon using a rigid instrument. In hip joint surgery, the field of access is limited by the relatively small clearance between the acetabulum and the femoral head which has been distracted or pulled somewhat out of the acetabulum. Distraction of the femoral head from the hip joint is necessary to provide the physician with access to the joint surfaces. Once the femoral head is separated from the hip joint, access to various surface aspects of the hip joint and femoral head requires controlled movement of the patient's leg in a full range of motion and fixation of the leg in selected positions. However, there is a limit to the surgeon's access to parts of the hip joint site from a given incision. Typically, the field of view is circular and provided by a triangulated scope. In contrast, the surgical access is somewhat conical in shape and may be elliptically conical, depending on the freedom of movement of the endoscopic instrument and the tissues and structures with which contact is to be avoided. It is particularly important to avoid unnecessary contact with the femoral head to minimize injury to the cartilage lining, since cartilage tends to have very limited capability of healing.

### SUMMARY OF THE INVENTION

The present invention provides improvements in endoscopic surgery by the use of an instrument to enable a surgeon to visually estimate the limits of a field of surgical access within a field of view of an endoscopic surgical site.

An embodiment of the invention provides a laser sighting endoscopic instrument or endoscope incorporating a laser unit for attachment to an endoscopic instrument in alignment with an axis of a lumen within a cannula portion of the instrument. The endoscopic instrument may, for example, be a portal instrument including a proximal hub with an elongated cannula extending therefrom. A lumen is formed through the hub and cannula toward a distal tip of the cannula. The hub has a socket formed at a rear port thereof which is configured to removably receive a self-contained laser unit. In one embodiment, the socket is threaded and a plug end of the laser unit is provided with complementary threading to enable the laser unit to be threaded into the hub of the portal instrument. The hub and laser unit, when joined, cooperate to position a laser beam from the laser unit along the longitudinal axis of the cannula. Alternatively, other types of junctions between the laser unit and portal hub are foreseen.

In one embodiment of the invention, the laser unit is provided with a laser generating element such as a laser diode, a power source such as a battery, and a control switch. It is foreseen that the laser unit could alternatively be powered by an external power source with a cable extending into the laser unit housing. The control switch can be a momentary switch for momentary activation of the laser by the surgeon or a latching or toggle type of switch which activates on a first press and deactivates on the next press of the button. The laser source is of such a character that the laser beam emitted therefrom is in the visible spectrum and bright enough for observation by the surgeon but low powered to avoid any heating of or other effects on tissues within the surgical site. It is also foreseen that the laser sighting endoscope can be used in conjunction with or incorporate a higher powered surgical laser unit to perform laser surgical procedures at the endoscopic surgical site.

Various objects and advantages of the present invention will become apparent from the following description taken in conjunction with the accompanying drawings wherein are set forth, by way of illustration and example, certain embodiments of this invention.

The drawings constitute a part of this specification, include exemplary embodiments of the present invention, and illustrate various objects and features thereof.

### BRIEF DESCRIPTION OF THE DRAWINGS

FIG. 1 is top plan view of an embodiment of a laser sighting endoscopic instrument or endoscope according to the present invention.

FIG. 2 is side elevational view of the laser sighting endoscope, shown partially in cross section and showing a diagrammatic cross sectional view of a laser unit for the instrument.

FIG. 3 is a fragmentary side elevational view at a somewhat enlarged scale and shows an internally threaded rear port of a hub of the endoscope to threadedly receive a laser unit therein.

FIG. 4 is a top plan view of an embodiment of a laser unit for use in the laser sighting endoscope.

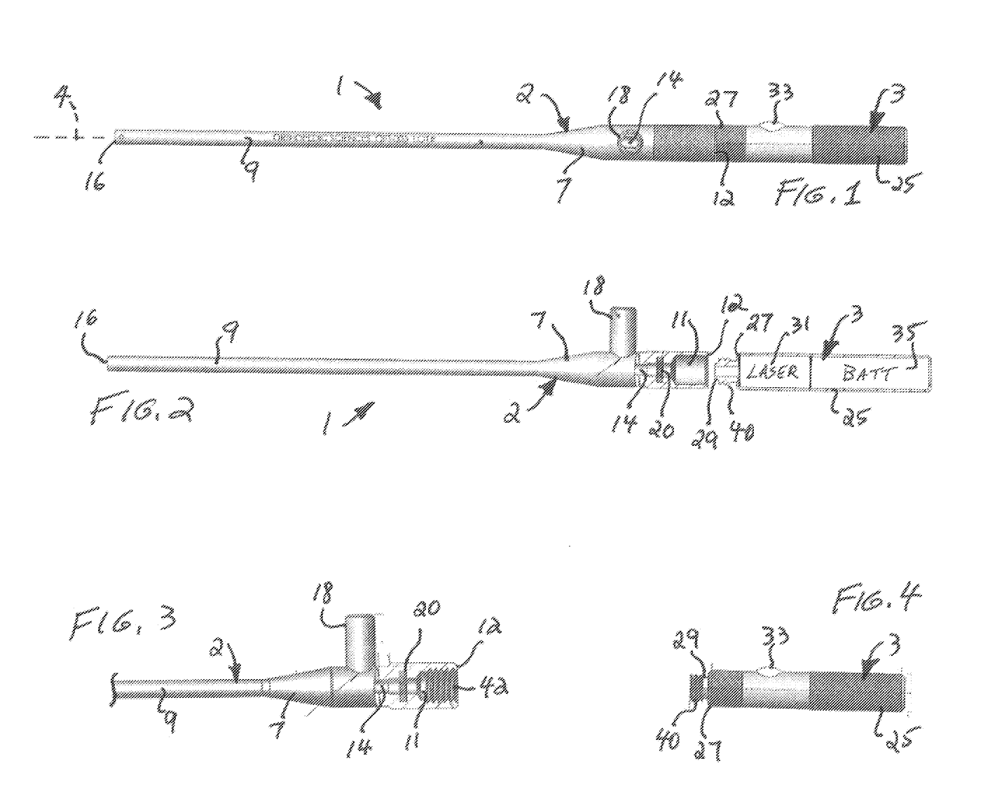

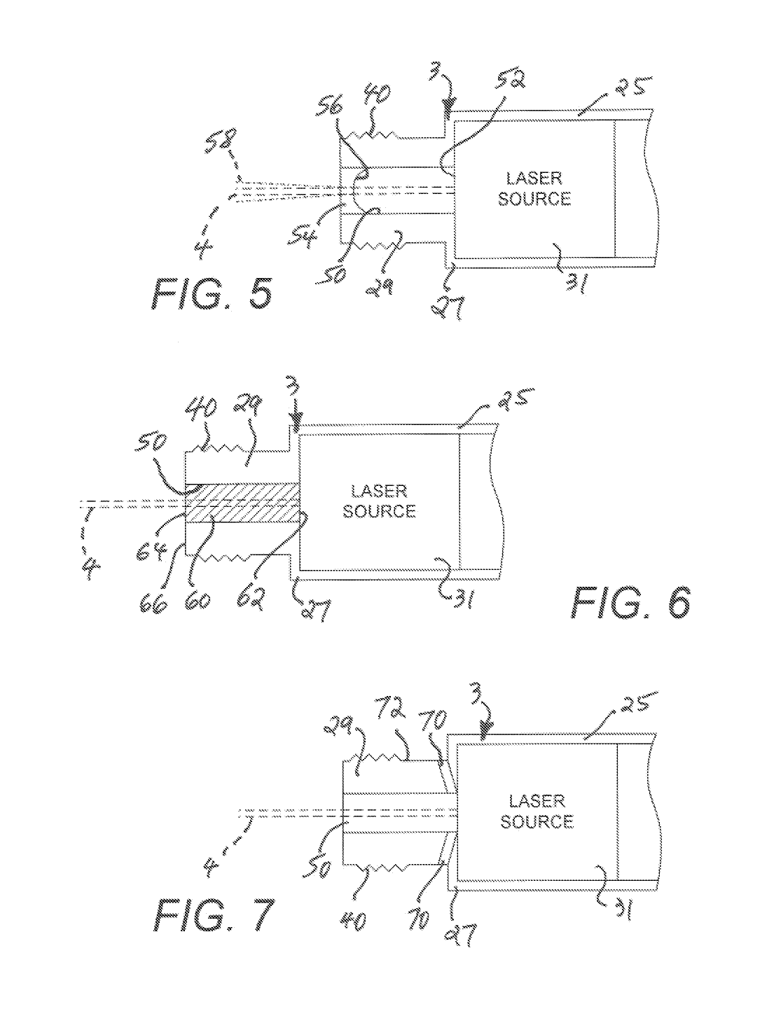

### DETAILED DESCRIPTION OF THE INVENTION

As required, detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

Referring to the drawings in more detail, the reference numeral 1 generally designates an embodiment of a laser sighting endoscopic instrument or endoscope according to the present invention. The instrument 1 generally includes an endoscopic instrument or endoscope unit 2 and a laser unit 3 removably joined with the endoscope unit 2 for sending a laser beam 4 therethrough.

The illustrated endoscopic unit 2 includes an enlarged hub or grip section 7 at a rear end from which an elongated rigid cannula 9 extends. The hub 7 has an enlarged passage 11 terminating proximally in a rear port 12 and which also communicates with an elongated opening or lumen 14 which communicates with the passage 11 and extends through the remainder of the hub 7 and the cannula 9 to a distal end 16 of the cannula 9. Optionally, the hub 7 may include a side port 18 communicating with the lumen 14 or the passage 11. The hub 7 may be provided with one or more seal members or O-rings 20 to control the outflow of fluids from a surgical site through the rear port 12. The endoscopic unit 2 may be any type of endoscopic instrument. The illustrated unit 2 is configured as a portal instrument which is employed to establish and maintain an open path from an incision to a surgical site. Portal instruments also provide for the insertion of endoscopic instruments toward the surgical site and are used to manage the introduction and removal of various fluids to and from the surgical site. As a portal instrument, the distal end 16 of the cannula 9 of the illustrated endoscopic unit 2 has a non-cutting circular edge.

The illustrated laser unit 3 includes a laser unit housing 25 which terminates at a distal end 27 in an attachment section or plug 29. The housing 25 has a laser source 31 (shown diagrammatically as “LASER” in FIG. 2) such as a laser diode which generates the beam 4. The laser source 31 may include further circuitry, including a control or activation switch 33, and may be powered by a power source or battery (BATT) 35 carried in the housing 25 or by an external power supply. The control switch 33 may be a momentary push-button type of switch which causes the laser source 31 to activate as long as the switch 33 is held closed or may be a latching toggle type of switch in which a first momentary operation of the switch 33 activates the laser source 31 and a next operation of the switch deactivates the laser source 31. In the illustrated laser unit 3, the attachment section or plug 29 is configured as a threaded barrel having threads 40 which are configured to mate with complementary threads 42 (FIG. 4) formed in the enlarged passage 11 of the hub 7. It is foreseen that other types of junctions between the laser unit 3 and hub 7 of the endoscope unit 2 could be employed, such as a frictional fitting, a snap-in arrangement, a key and groove arrangement, a bayonet connection, a Luer fitting, or the like.

The illustrated laser unit 3 is similar in many respects to the types of laser units that are used as pointing lasers, as for use in presentations. However, the laser unit 3 is preferably smaller in overall size for convenient use with the endoscope unit 2. Such pointing lasers generate a thin beam of coherent monochromatic light and typically have a laser power output in the range of about one 1 to 5 mw (milliwatts). The laser unit 3 preferably has a laser power output at the low end of such a range to avoid any heating or other effects on tissues at the surgical site. Pointing types of lasers are available in a number of colors. For a given level of laser power, green lasers having a wavelength of about 532 nm (nanometers) appear brightest because the typical human eye is most sensitive to light in the green region of the visible spectrum. Although a 532 nm green laser source 31 is preferred in the laser unit 3, it is foreseen that other color lasers could be employed. Because even low power laser devices can cause injuries, especially to the eyes, the manufacture and approval of such devices is regulated by government agencies.

In use of the laser sighting endoscope 1, the endoscope unit 2 is inserted through an incision toward a surgical site, such as a hip joint at which a femoral head has been distracted from an acetabulum of the patient's pelvic bone. A viewing scope with a light source (not shown) may be inserted through a separate incision to provide a visual image of the surgical site. The laser unit 3 may be attached to the endoscope unit 2, as by insertion of the attachment section 29 into the enlarged passage 11 and mating the threads 40 and 42. Typically, the visual field available to the surgeon greatly exceeds the field of reach or access using an endoscopic instrument with a rigid cannula. That is, the surgeon can see regions within the surgical site which cannot be reached for surgical operations using the rigid endoscope. In order to determine and visualize the actual field of surgical access, the surgeon activates the laser unit 3 by operation of the switch 33 to thereby radiate a laser beam through the lumen 14 of the cannula 9 into the surgical site. The surgeon can then manipulate the endoscope unit 2 to determine the available degree of freedom of the endoscope unit 2. Additionally, the surgeon can visually note any potential contact with sensitive tissues, such as femoral head cartilage, by illumination of the laser beam 4 without actual contact with such tissues. Once the surgeon has a feel for the prudent field of surgical access, the laser unit 3 can be removed from the endoscope unit 2 and replaced with various surgical tools for carrying out surgical operations such as cutting, shaving, debriding, cauterizing, or the like.

It is foreseen that the sighting laser unit 3 can be replaced with a surgical laser unit (not shown) for required surgical operations. Laser units employed for surgeries tend to be much higher powered, such as in the range of about 30 to 100 watts. It is also foreseen that such a surgical laser unit could be combined with a sighting laser unit 3 with optical elements, such as a prism or prisms, employed to direct the beams therefrom through the lumen 14.

It is also foreseen that if the laser beam 4 is not aligned substantially with the axis of the lumen 14, impingement of the beam 4 with internal surfaces of the lumen 14 can cause some decollimation or dispersion of the laser beam 4. Because of the relatively short distance involved in the length of the cannula 9 and the distance from the tip 16 to tissues within the surgical site, such dispersion would not be detrimental to the function of the instrument 1. The instrument 1 could still be used to effectively determine the field of surgical access at the surgical site.

It is to be understood that while certain forms of the present invention have been illustrated and described herein, it is not to be limited to the specific forms or arrangement of parts described and shown.

The inventor hereby states his intent to rely on the Doctrine of Equivalents to determine and assess the reasonably fair scope of the present invention as pertains to any apparatus not materially departing from but outside the literal scope of the invention as set forth in the following claims.

Having thus described the invention, what is claimed as new and desired to be secured by Letters Patent is as follows:

1. A laser sighting endoscopic instrument for visually observation of a field of surgical access within an endoscopic surgical site, said laser sighting endoscopic instrument comprising:
an endoscopic instrument having a proximal hub with an elongated cannula extending therefrom;
a laser unit in alignment with said cannula;
a lumen formed through said proximal hub and cannula toward a distal tip of said cannula, said lumen adapted for receiving surgical instruments therethrough; and
said hub having a socket formed at a rear port thereof configured to removably receive said laser unit.

2. The laser sighting endoscopic instrument of claim 1 wherein said endoscopic instrument in receipt of said laser unit form a laser beam along a longitudinal axis of said cannula.

3. The laser sighting endoscopic instrument of claim 1 further comprising a threaded receiver associated with one end of said cannula receiving one end of said laser unit associated with a helical thread.

4. The laser sighting endoscopic instrument of claim 1 wherein said laser unit has a momentary switch for one fingered activation of a laser between a on and an off position.

5. A method of visually observing a field of surgical access from a field of view of an endoscopic surgical site, said method including the steps of:
inserting a laser sighting endoscope with a laser unit attached to an endoscopic instrument, through an incision toward a surgical site,
emitting a laser beam from said laser unit axially along a lumen associated with the endoscope instrument;
visualizing the field of surgical access through operation of a switch associated with said laser unit whereby said laser beam radiates through the lumen of a cannula into the surgical site;
manipulation of the endoscopic instrument to determine the available degree of freedom from contact with selective tissues;
removal of said laser unit from said endoscopic instrument; and
selective receipt of desired surgical instruments through said lumen into the surgical site.
